(19) United States
(12) Patent Application Publication
(10) Pub. No.: US 2006/0095056 A1
(43) Pub. Date: May 4, 2006

(54) Device for incising a blood vessel

(76) Inventors: Peter Douglas, New Milford, NJ (US); Willem Suyker, Zwolle (NL); Mark Howansky, Union City, NJ (US); Clifford Volpe, Riverdale, NJ (US); Paul Suyker, Amsterdam (NL); Simon Cohn, Rutherford, NJ (US); James Rudnick, Mahwah, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/978,317

(22) Filed: Oct. 29, 2004

(51) Int. Cl. A61B 17/22

(52) U.S. Cl. 606/159

(57) ABSTRACT

A device for incising a blood vessel includes a handle having a longitudinal axis, a stationary blade having an upper surface and extending from the distal end of the handle at an angle out of line with respect to the longitudinal axis, and a moving blade having a distal portion that includes a leading edge. The moving blade is slidable with respect to the stationary blade. The distal portion of the moving blade is substantially aligned with the stationary blade. The moving blade has a first position and is movable to a second position to cut tissue disposed between the upper surface of the stationary blade and the leading edge of the moving blade.

### BACKGROUND OF THE INVENTION

1. Field of Invention

The invention relates to the field of medical instruments, and more particularly, to a device for creating an incision in a hollow lumen, such as an artery or vein.

2. Description of Related Art

In many surgical procedures, a surgeon must make a substantially linear incision in a hollow structure having a lumen, such as a blood vessel. For example, the creation of an incision is generally the first step in creating a new blood flow path that bypasses a blockage or stenosis within an artery. In such a bypass procedure, a graft vessel, which can be a vein or an artery or a synthetic tube, is connected or anastomosed to the target vessel downstream the blockage or stenosis. The graft vessel acts as a conduit to take blood from its natural, unobstructed origin, and permit it to flow through the anastomosis to the target vessel at a location downstream of the original obstruction. Alternatively, the graft may be severed from its natural origin, and may be anastomosed to another, big blood vessel such as the aorta to take blood from. Where the bypassed vessel is a coronary artery, the procedure is known as coronary artery bypass graft (CABG) surgery. The connection made at the aorta is referred to as the proximal anastomosis and the connection or connections made at the coronary artery downstream of the obstruction is referred to as the distal anastomosis. The anastomosis can be end-to-side, requiring a side hole, generally made by a precise incision in the target vessel only, or can be side-to-side, requiring matched incisions in both the target vessel and the graft vessel.

A successful bypass graft creates blood flow to a previously blocked or substantially blocked artery. To maintain the new flow path, the anastomosis or connection between the graft vessel and the coronary or target vessel, must provide a smooth transition from the graft vessel to the target vessel. A poorly created incision may result in loose intimal flaps that create turbulence and obstruction with secondary thrombus formation at the anastomosis site, which in turn induces smooth muscle cell migration to the site as part of the body healing response. This healing response may lead to stenosis or a blocking of the anastomosis and associated artery. In addition, the incision must completely penetrate a portion of one side of the wall of the artery to create an opening without damaging any other tissue, such as the back wall of the artery near the site of the incision.

Further, the incision needs to be straight, uniform and of a defined length as the opening created by the incision is sized to communicate with the inner lumen of the graft vessel that the surgeon connects to the opening in the coronary vessel. This is particularly true when the surgeon uses an automatic anastomosis device to facilitate the creation of the anastomosis. In such a procedure, rather than hand sewing the graft vessel to the target vessel, the surgeon uses an anastomosis device or connector to make the connection. These connector devices can provide the benefit of a quicker, and potentially more reliable anastomosis, than a hand sewn anastomosis, even under limited access conditions. However, such connector devices are sized for a particular graft and target vessel and, as such, a particular incision length. Thus, it will be clear that a uniform, quality incision is desirable, whether the anastomosis is hand sewn or created with a connector, but an incision with a precisely defined, consistent length is even more desirable when the anastomosis relies on a connector.

The standard method for creating an incision in a cardiac vessel requires that the surgeon first pierce the vessel with a small scalpel to create a stab wound, for example using a scalpel having a 15 degree tip, like a Sharpoint® scalpel (Sharpoint Inc., Reading Pa.). The surgeon then may push the scalpel into the vessel lumen and enlarge the stab wound by cutting the vessel wall with the scalpel blade using an inside-out motion. During this step, the surgeon has to take particular care not to damage the back side of the vessel (referred to as “backwalling”). The surgeon then typically uses micro scissors to cut the arteriotomy to a desired length by extending the initial incision in one or both directions.

The creation of a uniform incision of a defined length is a difficult task when the surgeon uses a scalpel and micro scissors. As it is micro scissors generally do not make incisions of a consistent quality across the length of the incision they create. Practically, the incision created in the tissue cut near the tip of the micro scissors may often not be the same as the quality of the incision in the tissue cut near the pivot point of the micro scissors. Specifically, tissue cut at the tip of the micro scissors may be crushed, rather than neatly cut. This problem is caused at least in part because the cutting angle between the jaws of the scissors gradually decreases to almost zero near the tip as the cutting edges of opposing scissor blades assume a near parallel position during the cutting action, an issue intrinsically related to the pivotable nature of how a pair of scissors works. A small cutting angle stresses the mechanical parts of the micro scissors, and can lead to a failure to cut tissue.

To further complicate the procedure, where the surgery is performed on a beating heart, the surgical field is small, creating access issues that make it difficult for the surgeon to precisely manipulate the instruments, especially when attempting to anastomose to an artery on the posterior or inferior wall of the heart. Surgeons also do not typically have an accurate means of measuring the required arteriotomy size so it is difficult to precisely cut the intended length. Surgeons therefore rely on their subjective estimation of the desired arteriotomy length. The length of an arteriotomy created in this manner has been shown to be highly variable and inaccurate.

Where the surgeon determines that the length of the initial incision is too short, the surgeon will be required to lengthen the incision by again using micro scissors or a scalpel to cut the tissue. The use of these types of tools a second time creates the possibility that the resulting incision will not be aligned with the initial incision along with the attendant deficiencies of using the micro scissors discussed above.

Where a surgeon uses an anastomosis device, if the surgeon determines that the length of the incision is too long while performing an end-to-side anastomosis or a side-to-side anastomosis, then the surgeon can add additional stitches to close the remaining incision around the anastomotic device. This type of repair causes its own problems, as any additional suturing devaluates the benefit of an automated anastomotic system, requires access for carefully manipulated instruments, and last, but not least, may increase the likelihood of stenosis, due to a reduction in diameter or due to the body's healing response to an injury.

Where a surgeon hand sews an anastomosis, if the surgeon determines that the length of the incision is too long, especially while performing a side-to-side anastomosis where the graft vessel and the target vessel cross one another at an angle of 90° (a “diamond-shaped anastomosis”) and the graft vessel is anastomosed to more than one coronary artery (a “jump graft”), then the problem is more critical. Where this occurs, the surgeon is left with a difficult decision. Either suture the incision to shorten it and thereby risk diameter reduction and stenosis. Or, alternatively, connect the graft at the overly long incision, and risk that the graft vessel may flatten to accommodate the lengthy incision (the “seagulling” phenomenon), which may cause the graft vessel to effectively close off at the anastomosis site, thus putting both the current and all existing downstream both the current and all existing at risk.

### BRIEF SUMMARY OF THE INVENTION

The present invention addresses the shortcomings of the prior art by providing an improved device for creating an incision.

The described device is designed to create reliably and consistently a quality. arteriotomy or venotomy, or incision in any other tubular natural or synthetic structure, of a defined length. An arteriotomy is an incision in the wall of an artery reaching the lumen, while a venotomy is such an incision in case of a vein. While the device is designed for use in human coronary arteries and arterial as well as venous bypass grafts during CABG, those skilled in the art understand the embodiments described herein have broader application in creating an incision in any hollow tissue structure, such as the intestines, the bladder, the ureter, other types of blood vessels, or other similar tubular structures.

According to the present invention, an device for incising a blood vessel includes at least one gripping portion, a stationary blade attached to the gripping portion that has a neck and a foot connected to the neck. The foot has a cutting edge along an upper surface. The device includes a relatively blunt moving blade having a leading edge and which is operatively movable with respect to the stationary blade. The moving blade has a first position, proximal to the upper surface of the stationary blade, and is movable to a second position, distal to the upper surface of the stationary blade, to cut tissue disposed between the upper surface of the stationary blade and the leading edge of the moving blade.

A method for making an incision in the wall of a hollow tissue structure that has an outer surface, an inner surface and a lumen, includes the steps of: (a) providing a device having a first blade, the first blade having a cutting edge along an upper surface, and a relatively blunt second blade having a leading edge, the moving blade being movable relative to the first blade from a first position, where the leading edge of the second blade is proximal to the upper surface of the first blade, to a second position, where the leading edge of the second blade is distal to the upper surface of the first blade; (b) incising the outer surface to create a small incision in the wall; (c) passing at least the cutting edge of the first blade through the small incision and into the lumen; and (d) creating a larger incision in the wall by using the first blade in cooperation with the second blade to cut from the inner surface to the outer surface when the second blade moves from the first position to the second position.

### BRIEF DESCRIPTION OF THE DRAWINGS

These and other features, advantages and benefits will be made apparent through the following descriptions and accompanying figures, where like reference numerals refer to the same features across the various drawings.

FIG. 1 is a perspective view of a device for incising a blood vessel according to the present invention;

FIG. 2 is an exploded view of the device for incising a blood vessel of FIG. 1;

FIGS. 3(a)-(c) are, respectively, a top plan view, a side cross-sectional view of FIG. 3(a) taken along line 3(b)-3(b), and an end view of the stationary blade of the device for incising a blood vessel of FIG. 1;

FIG. 4 is the preferred embodiment of the foot of the stationary blade of the current invention;

FIGS. 5(a)-5(c) are alternate embodiments of the feet in accordance with the current invention;

FIGS. 6(a) and 6(b) are bottom views of the stationary blade and moving blade of vessel incisor of the current invention (not shown to scale so as to more clearly demonstrate the concepts);

FIG. 7 is a side view of another embodiment of a stationary blade foot;

FIGS. 8(a) and 8(b) are schematic views of the use of stationary blade depicted in FIG. 7;

FIG. 9 is a cross-sectional view of the device for incising a blood vessel of FIG. 1 in a first configuration;

FIG. 10 is a perspective view of the distal portion of the device for incising a blood vessel of FIG. 9 in the first configuration;

FIG. 11 is a detailed cross-sectional view of the device for incising a blood vessel of FIG. 1 in a second, cutting configuration;

FIG. 12 is a perspective view of the distal portion of the device for incising a blood vessel of FIG. 11 in the second configuration;

FIGS. 13(a)-13(d) are schematic views of the operation of the distal end of the vessel incisor according to the present invention;

FIG. 14 is a cross-sectional view of an alternate embodiment of the vessel incisor of the current invention;

FIGS. 15(a) and 15(b) are cross-sectional views of an alternate embodiment of the vessel incisor of the current invention;

FIG. 16 is a perspective view of another alternate embodiment of the vessel incisor of the current invention;

FIG. 17 is a perspective view of yet another alternate embodiment of the vessel incisor of the current invention;

FIGS. 18(a) and 18(b) are schematic views of embodiments of the vessel incisor of the current invention, each having a malleable portion;

FIG. 19 is a perspective view of an alternate embodiment of a device for incising a blood vessel having a rotatable distal handle portion; and

FIG. 20 is a side cross-sectional view of the device of FIG. 19.

### DETAILED DESCRIPTION OF THE INVENTION

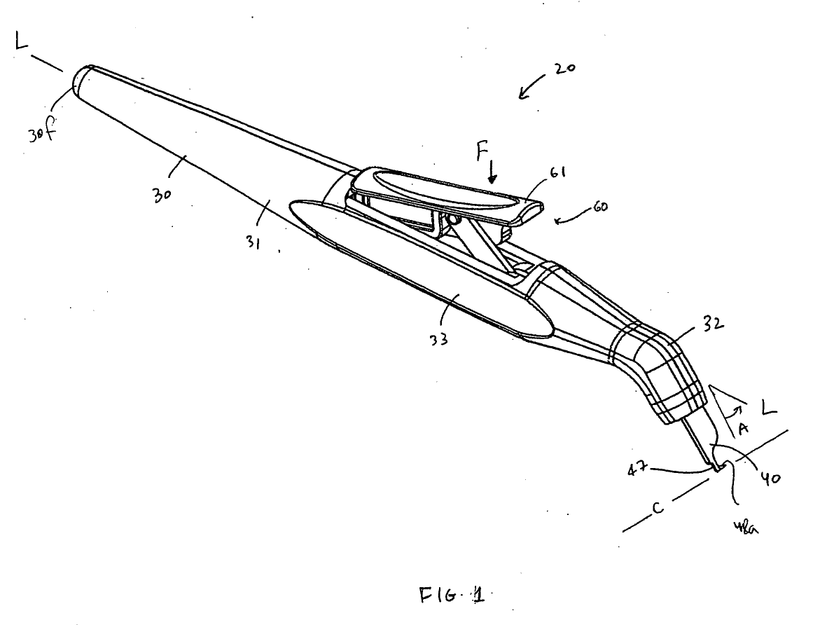

FIG. 1 illustrates a perspective view of one embodiment of a device for incising a blood vessel of the current invention. The vessel incisor is indicated generally as reference numeral 20. The vessel incisor 20 is a hand-held instrument that includes a handle or housing 30, a stationary blade 40, a moving blade 50 and an actuator 60 for moving moving blade 50 relative to stationary blade 40.

Handle 30 has a proximal portion 31 designed to be grasped by the user and a distal portion 32 extending from the proximal portion at an angle A defined relative to a longitudinal axis L defined by the proximal portion of the handle. For the purposes of this application, “proximal” is defined as being closer to the surgeon's hand and “distal” is defined as being closer to the blood vessel incision site. Angling the distal portion 32 away from proximal portion 31 of handle 30 provides the surgeon with better visualization of the incision site when using vessel incisor 20. Preferably, distal portion 32 of handle 30 extends from proximal portion 31 at an angle A that ranges from approximately twenty (20) degrees to approximately ninety (90) degrees, but most preferably angle A is approximately forty-five (45) degrees.

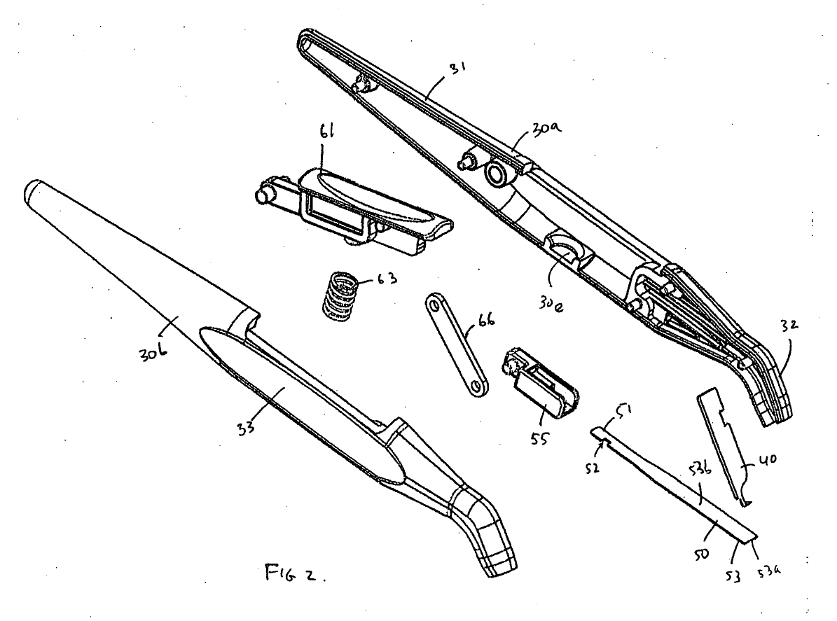

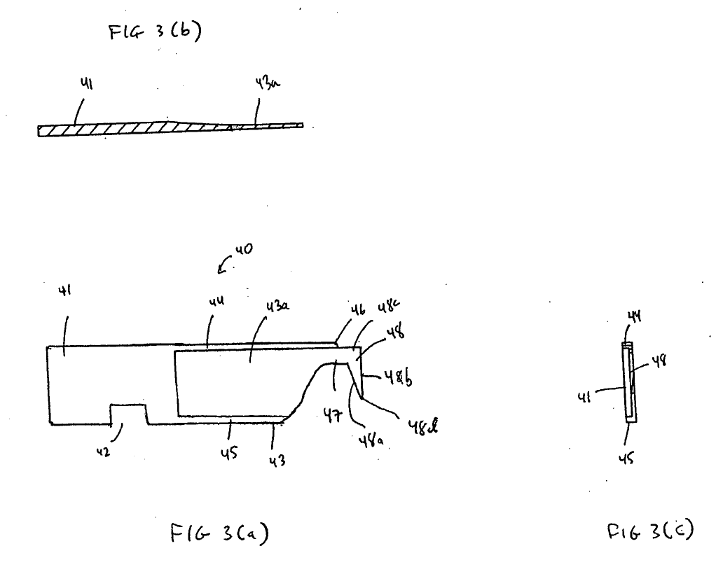

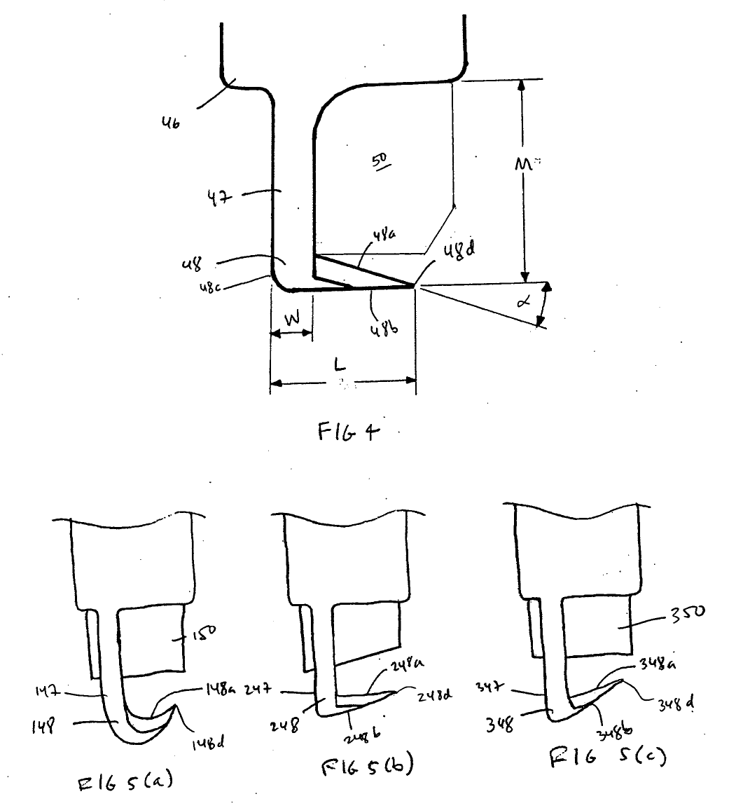

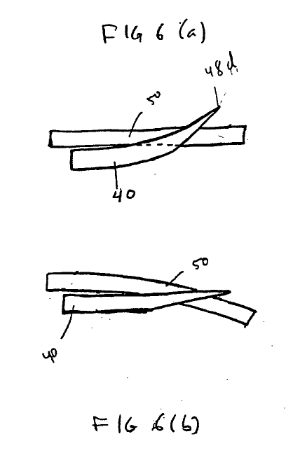

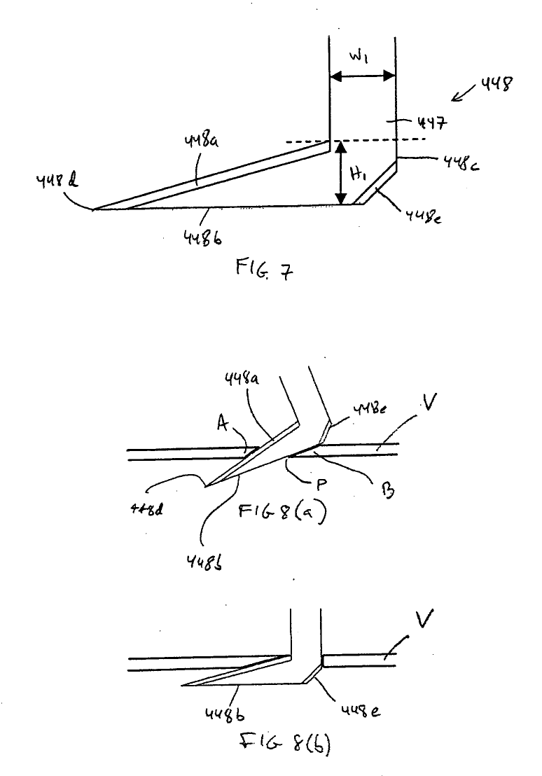

Referring to FIGS. 1 and 2, handle 30 is generally fabricated from a medical grade plastic and is preferably formed in a “clamshell” design having first and second halves 30a, 30b. The clamshell design allows for easy assembly of the internal components. The halves 30a, 30b are fixed together by any means known in the art, such as by a press fit, or with a medical grade epoxy or adhesive, or by ultrasonic welding or by mechanical means, such as by screws, or by any combination of the above. In a preferred embodiment, handle 30 includes flats 33 on either side of proximal portion 31 designed to accommodate a surgeon's thumb and middle finger when vessel incisor 20 is grasped by the surgeon. When halves 30a, 30b are assembled, they form an opening to accommodate actuator 60 (described in more detail below). Handle 30 is approximately 6.0 inches in overall length as measured from the tip of the cutting blade to proximal end 30f. The distance from the midpoint of the button to proximal end 30f is approximately 3.5 inches.

Vessel incisor 20 includes a stationary blade 40 for piercing the vessel wall and cutting the vessel wall from a position within the lumen. Stationary blade 40 is shown generally in FIGS. 1 and 2 and more particularly in FIGS. 3(a) through 3(c). Stationary blade 40 is formed of a thin sheet of metal, preferably stainless steel, and is fixed to, and preferably disposed at least partially within, distal portion 32 of handle 30. Stationary blade 40 has a proximal portion 41 and a distal portion 43. Proximal portion 41 has a notch 42 that is configured to mate with a molded structure within distal portion 32 of handle 30 to retain stationary blade 40 within distal portion 32. Referring to FIG. 3(b), distal portion 43 includes a thinned portion 43a that has a thinner thickness (shown in cross section) than proximal portion 41. As is shown more particularly in FIGS. 3(a) and 3(c), distal portion 43 includes guides 44 and 45 that extend orthogonally from thinned portion 43a along either side of blade 40. Preferably, guides 44 and 45 are formed with a height that is greater than the thickness of moving blade 50. As such, as moving blade 50 moves distally with respect to stationary blade 40, moving blade 50 is constrained to move between guides 44 and 45.

Referring to FIG. 3(a), distal portion 43 of stationary blade 40 includes a neck 47, that extends distally from one side of thinned portion 43a and is narrower in width than thinned portion 43a. A shoulder 46 extends partially along one side of neck 47, and a foot 48 extends away from the other side of neck 47. Foot 48 includes a cutting edge or surface 48a for cutting tissue, a bottom edge or surface 48b and a heel 48c. Foot 48 also includes a sharp tip 48d for piercing the wall of the vessel. The surfaces of the bottom edge 48b and neck 47 are blunt so as to prevent the surgeon from unintentionally cutting tissue with these surfaces during the piercing or cutting action. In the embodiment shown in FIG. 3(a), the surface of the heel is also blunt.

Referring to FIG. 4, in a preferred embodiment, the length M of neck 47 may range from 3 mm to 7 mm in length, but preferably is approximately 5 mm in length. The length L of foot 48 is designed to match the length dictated by the anastomosis device or connector used to connect the graft vessel with the target coronary vessel. In a preferred embodiment, the length L of foot is between 3 mm and 5 mm, but preferably is approximately 4 mm. Preferably, the width W of neck 47 is approximately 1 mm. Cutting surface 48a preferably extends from bottom surface 48b at a cutting angle α that ranges from 14 degrees to 24 degrees, but is most preferably 19 degrees. Moving blade 50 is shown in a partially actuated position to illustrate that cutting angle α remains constant as the leading edge of moving blade 50 is moved toward cutting edge 48a of stationary blade 40. Thus, unlike the cutting angle of a pair of micro scissors which decreases as the pair of blades pivot toward one another, the cutting angle of device 20 remains constant through the cutting length of cutting edge 48a, thereby cutting the end point tissue as reliably as the starting point tissue.

Shoulder 46 preferably has a width of approximately 0.5 mm, but in any event should be of a width that provides resistance to distal movement to the surgeon when neck 47 is disposed within the vessel puncture. In this way, shoulder 46 butts up against the adventitia or outer surface of the blood vessel and prevents foot 48 from unnecessarily contacting the inner surface of the blood vessel. As is shown in FIG. 4, heel 47 can be rounded so as to help guide foot 48 through the initial puncture created by tip 48d.

Referring to FIG. 5(a) through 5(c), alternate configurations of foot 48 are shown formed to prevent tissue disposed proximally relative to foot 48 from sliding off of cutting edge 48a. For example, foot 148 shown in FIG. 5(a) is formed in an arcuate shape so as to capture tissue in a trough 148a of the arc. In the alternative, as is shown in FIGS. 5(b) and 5(c), foot 248 can have a bottom surface 248b that is not orthogonal to neck 247 (as is the case in the embodiment depicted in FIG. 4). Instead, bottom surface 248b, 348b can extend at an acute angle relative to neck 247, 347, respectively. Cutting surfaces, in these embodiments, may extend either orthogonally from neck 247, as is the case with surface 248a in FIG. 5(b), or at an acute angle from neck 347, as is the case with surface 348a in FIG. 5(c). As will be understood to one skilled in the art, the features described above may be utilized in combination with one another

To more reliably cut tissue between moving blade 50 and cutting blade 40, the embodiments of vessel cutters 20 depicted in FIG. 4 and vessel cutter 220 in FIG. 5b may be useful as tissue cut with these embodiments is cut starting at neck 47, 247 and the cutting continues in a direction away from neck 47, 247 toward tip 48d, 248d. Embodiments such as those shown in FIGS. 5(a) and 5(c) cut tissue from tip 148d, 348d and continue cutting tissue toward neck 147, 347 as moving blade 150, 350 is moved distally.

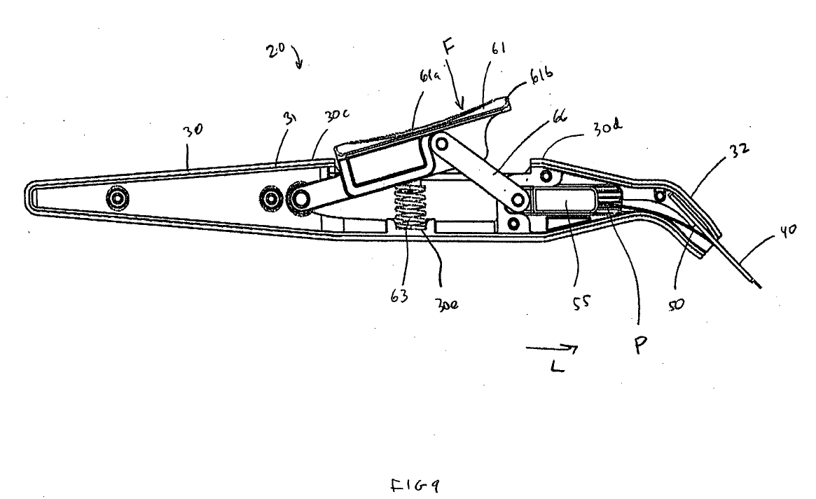

Vessel incisor 20 also includes moving blade 50 that moves relative to stationary blade 40 to cut tissue disposed between moving blade 50 and stationary blade 40. The shape of moving blade 50 is a much less complex than the shape of stationary blade 40. Referring to FIGS. 2 and 8, moving blade 50 includes a proximal end 51, having a notch 52, and a distal end 53. Moving blade 50 is preferably between 1.5 and 2.0 inches in length, and most preferably approximately 1.75 inches in length. The width of moving blade 50 is preferably slightly less than the distance between guides 44 and 45 of stationary blade 40. In a most preferred embodiment, moving blade 50 is approximately 0.20 inches at its widest measurement and has a substantially uniform thickness of approximately 0.012 inches. Notch 52, like notch 42 of stationary blade 40, is configured to mate with a molded structure of a slide 55 (described below) within handle 30 to retain stationary blade 40 within handle 30.

Distal end 53 preferably has a relatively blunt leading edge 53a, which is preferably ground and polished to have an angle that ranges from about 20° to about 5°, but is most preferably approximately 10° (relative to a standard flat or leading edge). The point of leading edge 53a is positioned nearer to bottom surface 53c than top surface 53b such that leading edge 53a of moving blade 50 resists and compresses tissue disposed between leading edge 53a and cutting edge 48a of stationary blade 40 to permit cutting edge 48a to cut or shear the tissue.

To maximize the effectiveness of the cut, leading edge 53a of moving blade 50 and cutting edge 48a of stationary blade 40 must maintain good point contact along the length of the cut. In one embodiment, depicted in FIG. 6(a), good point contact is achieved by applying a slight bias or bend to tip 48d of stationary blade 40, so that stationary blade 40 flexes against moving blade 50 during the entire stroke. Alternatively, in a preferred embodiment depicted in FIG. 6(b), moving blade 50 could be biased or bent to stationary blade 40. In yet another alternative, at least the distal end of each blade 40, 50 may be biased against the other blade, or, further, leading edge 53a of moving blade 50 could have an angled grind pattern to mimic a bend. Further, moving blade 50 may be positioned within housing 30 such that when moved from the first position leading edge 53a contacts and is deflected by stationary blade 40 so as to be biased against stationary blade 40 prior to moving blade 50 reaching the second position (where leading edge 53a is distal of cutting edge 48a). One skilled in the art will understand that any one of these features in combination or alone may be used to ensure that there is a good point contact when tissue is cut between blades 40, 50.

In use, when the surgeon uses device 20, and moving blade 50 is moved relative to stationary blade 40 between guides 44, 45, leading edge 53a presses tissue against cutting edge 48a of stationary blade 40, which cuts tissue disposed between the two blades with a shearing motion. Because cutting edge 48a of stationary blade 40 is sharper than leading edge 53a of moving blade 50, the stationary blade 40 cuts the vessel wall from the inside out, by cutting first the intimal layer of tissue, then the medial layer of tissue, and finally the tougher adventitial layer of tissue. In this way, the device first cuts the tissue on the inside of the blood vessel, which is softer than the tissue on the outer surface of the blood vessel. This is the preferred sequence from a surgical, as well as a biological point of view, since the vulnerable, delicate inner layers of the vessel are divided and fall aside before more force is required to cleave the much stronger outer layers. The “inside-out” cutting motion thus tends to avoid intimal crush and loose intimal flaps, which is important for anastomotic quality and the patency of the bypass graft.

Cutting the vessel wall from the inside out also greatly benefits from the use of the relatively blunt moving blade (or anvil) acting on the outside of the vessel, as this arrangement offers reliable tissue support during the cutting action, thereby minimizing stress on the surrounding vessel wall. In addition, by ensuring good contact between sharp stationary blade 40 and the anvil of moving blade 50 during the entire cutting action, optimal use of shearing force is made in addition to pure sharp cutting provided by cutting edge 48a, thereby increasing the quality of the cut and the reliability of device 20. The combination of a sharp blade and an anvil thus produces better cuts, without loose, frayed ends, as compared with a sharp blade used without an anvil.

Referring to FIG. 7, an alternative embodiment of the foot of device 20 is shown and designated as foot 448. Structures that are similar to prior embodiments are numbered similarly. As with the embodiments described above, foot 448 includes a neck 447, a cutting edge 448a, a bottom surface 448b, a heel 448c and a tip 448d. But unlike the embodiment depicted in FIG. 4, which has a rounded heel, foot 448 includes a secondary cutting edge 448e generally located at heel 448c. Preferably, secondary cutting edge 448e extends from neck 447 to bottom surface 448b.

Referring to FIG. 8(a) foot 448 is depicted puncturing vessel wall V with tip 448d. The surgeon punctures vessel wall V by rotating device 20 such that tip 448d is positioned to contact vessel wall V. A puncture P is created by pressing tip 448d through vessel wall V. As tip 448d pierces vessel wall V, the surgeon rotates device 20 to move tip 448d and the distal portion of foot 448 through puncture P. As foot 448 is rotated, cutting edge 448a either cuts or distends tissue A proximate cutting edge 448a to widen the initial puncture to permit foot 448 to pass through puncture P. Recall that bottom surface 448b does not cut vessel wall V as it is a blunt surface. Due to the angle at which foot 448 enters the lumen relative to vessel wall V, foot 448 creates an angled, non-full-thickness starting point of the arteriotomy, indicated as vessel wall tissue B. Thus, when foot 48 of the embodiment shown in FIG. 4 is used, the rounded heel 48c passes tissue B leaving the angled, non-full-thickness starting point intact. In contrast, as shown in FIG. 8(b), when foot 448 reaches the position depicted in FIG. 8(a), secondary cutting edge 448e cuts tissue B as it pivots to a position within the vessel lumen, leaving a substantially perpendicular, full wall-thickness starting point, or at the very least a reduced obliquity at the starting point of the puncture. Thus, when moving blade 50 is moved distally relative to cutting edge 448a (as is described in connection with the first embodiment below), tissue A is also cut to form a substantially perpendicularly relative to the outside vessel wall, thereby creating an arteriotomy with substantially perpendicular starting and end points.

As discussed above, it is important to ensure that device 20 creates an incision of a defined length. Referring again to FIG. 7, the width W₁ of neck 447 and the height H₁ of foot 448 are indicated. One variable in reliably creating an intended incision length is whether heel 48 is properly inserted into the vessel lumen. To facilitate proper insertion, heel 48 could be rounded as shown in the embodiment of FIG. 4. A second means for doing so is to size width W₁ of neck 47 (or neck 447) to be less than or equal to height H₁ of foot 48 (or foot 448). In this way, when foot 48 has been inserted such that tissue A contact neck 47 on the side proximate cutting edge 48a, i.e., when the thickest portion of foot 48 has passed into puncture P, heel 48c will rotate freely into puncture P, as the width of neck 47 is less than the height of the thickest portion of foot 48. A foot 48 with such a construction can also have a rounded heel 48c or contain a cutting edge 48e. A rounded heel 48c can also have a cutting edge that follows the rounded contour. The important consideration is that foot 48 should be designed to enter puncture P with minimal resistance so that the user does not jog or rotate foot 48 out of line with the initial small incision as this may cause the starting portion of the ultimate incision to tail off at an angle relative to that portion of the incision created by cutting edge 48a.

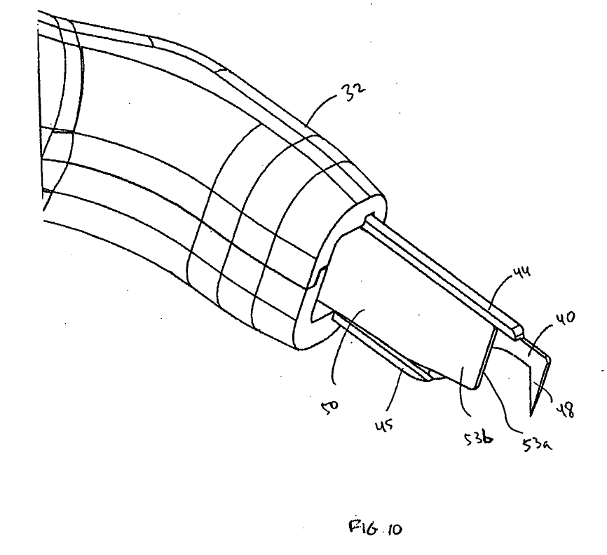

Vessel incisor 20 also includes actuator 60 for moving movable blade 50 relative to stationary blade 40. Actuator 60 includes a button 61 pivotably mounted to proximal portion 31 of handle 30 on a proximal end, a spring 63 disposed between button 61 and an inner surface 30e of handle 30, a slide 55 for moving in a longitudinal direction L, and a link 66 pivotably connected at a link proximal end to the distal end of button 61 and on a link distal end to slide 55. Referring to FIG. 9, slide 55 is slidable within a path P molded within handle 30 between a proximal position and a distal position. Slide 55 is configured to retain proximal end 51 of moving blade 50 such that slide 55 and moving blade 50 move together through path P.

In this manner, as shown in FIGS. 1 and 9, in a first position, spring 63 biases button 61 such that upper surface 61a of button 61 extends at an angle away from upper surface 30c of proximal portion 31 of handle 30. At this position, the distal end of link 66 is pulled to its proximal position, which in turn disposes slide 55 and moving blade 50 at their proximal positions. Referring to FIG. 10, at this proximal position leading edge 53a of moving blade 50 is positioned proximal of cutting edge 48a of stationary blade 40.

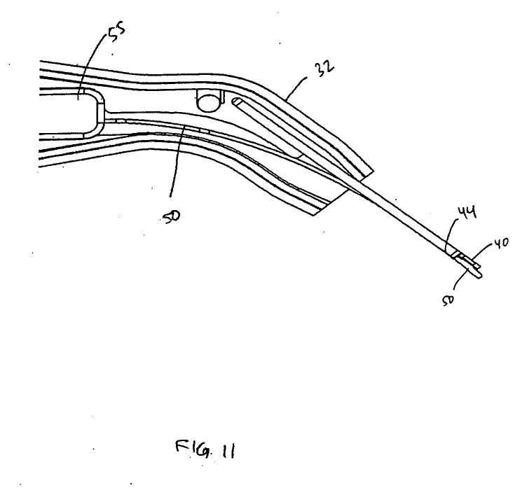

When the surgeon depresses button 61 against spring 63, the distal end 61b of button 61 pivots toward upper surface 30d of handle 30, thereby causing link 66 to pivot relative to button 61 and move distally within path P. This movement effectively lengthens link 66 relative to the longitudinal direction L, which in turn pushes slide 55 and moving blade 50 from their proximal positions to their distal positions through path P. Moving blade 50 is shown in its distal position in FIGS. 11 and 12. In FIG. 11, slide 55 is shown in its distal most position within path P, at which point leading edge 53a of moving blade 50 is located distal of cutting edge 48a of stationary blade 40. FIG. 12 is a perspective view of distal handle portion 32 with button 61 in the depressed position, whereat moving blade 50 extends distally beyond the distal end of stationary blade 40.

When the surgeon releases button 61, button 61 returns to its first position under the force of spring 63, thereby returning link 66 to its initial position, and slide 55 and moving blade 50 to their proximal positions. As slide 55 moves proximally, moving blade 50 also moves proximally with respect to stationary blade 40, thereby again ready to be used to create an arteriotomy.

An important consideration when using device 20 is to ensure that the distal end of is held steady when actuating moving blade 50. In this regard, handle 30 is designed to substantially separate the action of holding device 20 from the action of actuating device 20 so as to stabilize the instrument during actuation. Handle 30 permits the surgeon to hold device 20 in two positions to reduce or dampen tremors felt by the working or distal end of device 20 by stabilizing device 20 in the surgeon's hand. The surgeon may either hold device 20 with the palm of his or her hand in a prone (palm down) position or a supine (palm up) position. With reference to FIG. 1, when device 20 is employed with the palm of the hand in a prone position, proximal end 30f is designed to be of a length that the surgeon can rest proximal end 30f in the middle of his or her palm, or, alternatively, proximal end 30f can rest in the crook between the surgeon's thumb and forefinger. In either case, proximal end 30f is held as an extension of the hand, with the surgeon's hand facing the top surface of device 20, and the distal end of device 20 projecting from the surgeon's hand to a position beyond the surgeon's fingers. The thumb and middle finger each press against a separate one of flats 33 on either side of button 61, while the forefinger actuates button 61. In this position, the surgeon employs device 20 such that blades 40, 50 act at a location forward of the surgeon's hand.

When device 20 is held in the supine position, the surgeon employs device 20 such that blades 40, 50 act at a location between the surgeon's hand and body. As such, the surgeon has the added advantage of being able to clearly view the incision site while positioning the distal end of device 20. In particular, the surgeon can clearly view the target vessel while making the puncture and passing foot 48 of stationary blade 40 through the puncture and into the lumen of the vessel. In the supine position, the surgeon rests proximal portion 31 of handle 30 on either the index finger or both the index finger and middle finger with the palm facing the underside of the device, presses either the index finger and middle finger, or the middle finger and ring finger against a separate one of flats 33 on either side of button 61, and actuates button 61 with the thumb.

Whichever position the surgeon chooses, prone or supine, he or she may further stabilize device 20 by resting his or her arm on a stable point in the operating space, such as a retractor. In this manner, device 20 may be actuated while being held stably, thereby further minimizing the likelihood that the surgeon will inadvertently cut tissue.

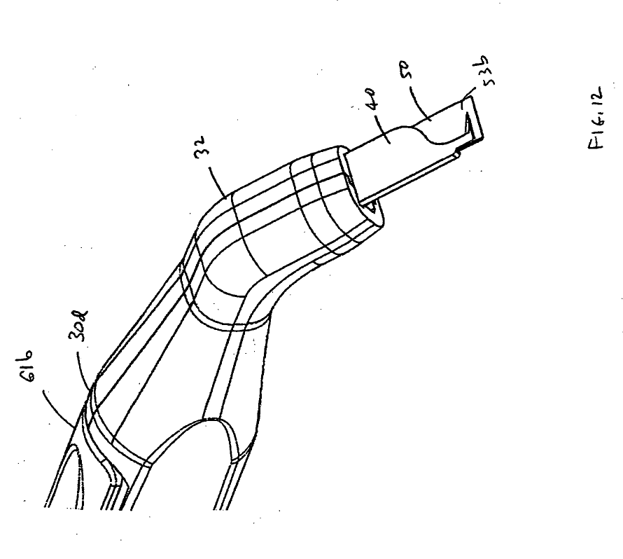

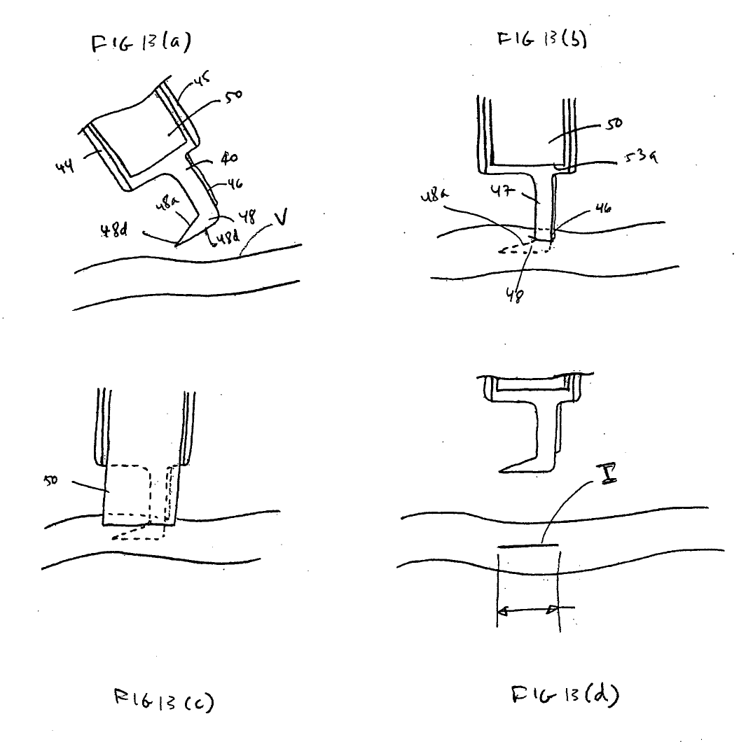

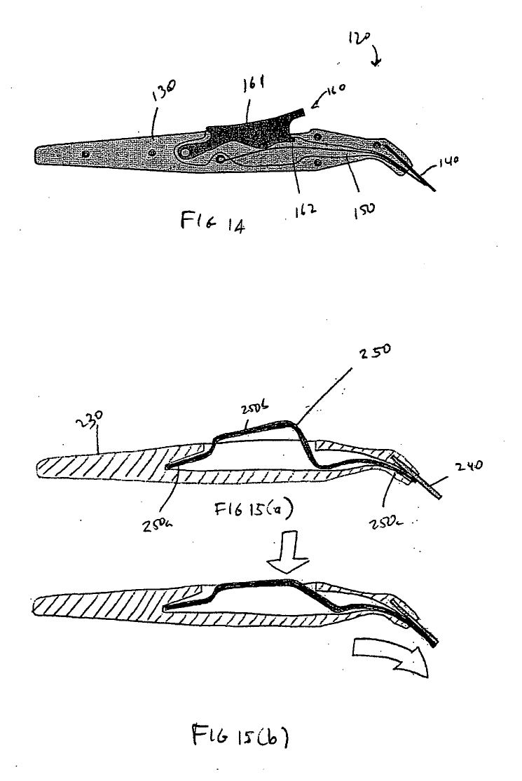

Referring to FIGS. 13(a) through 13(d), the steps of using device 20 to pierce and then cut the vessel wall V are depicted graphically. Once the surgeon determines an appropriate location on a vessel, the surgeon manipulates handle 30 to position distal portion 32 of device 20 with respect to the vessel. As is shown in FIG. 13(a), the surgeon then pierces vessel wall V with tip 48d of foot 48. When tip 48d pierces the vessel wall and passes into the vessel lumen, neck 47 is disposed within the puncture created by tip 48d. The tissue displaced by neck 47 will bunch or wrinkle toward cutting surface 48a.

Once foot 48 is within the vessel lumen, as depicted in FIG. 13(b), the surgeon rotates tip 48d relative to the inner surface of the vessel so as to position cutting blade 40 substantially orthogonally with respect to the vessel surface. Next, as is shown in FIG. 13(c), the surgeon depresses button 61 thereby actuating moving blade 50 and causing moving blade 50 to move relative to stationary blade 40. As leading edge 53a of moving blade moves toward cutting edge 48a of stationary blade 40, cutting edge 48a of stationary blade 40 cuts the tissue disposed between blades 40 and 50 from the inside out. An incision I is created from the backside of cutting edge 48a (nearest neck 47) to the distal tip 48d as moving blade compresses tissue disposed between stationary blade 40 and leading edge 53a of moving blade 50. Once the tissue disposed between tip 48d of stationary blade 40 is cut, stationary blade 40 no longer compresses tissue between cutting edge 48a and leading edge 53a of moving blade 50. As such, moving blade 50 no longer has a surface to act upon, and the cutting action ceases. At this stage, stationary blade has effectively exited the blood vessel lumen, and leading edge 53a of moving blade 50 is disposed distally of bottom edge 48d of stationary blade 40, leaving incision I, as shown in FIG. 13(d). The method is inherently safe as moving blade 50 never enters the blood vessel lumen. Rather, moving blade 50 remains in contact with the outer layer of the blood vessel wall during the cutting action.

As a result of the means by which the incision is formed, incision I created by vessel incisor 20 is largely independent of any measuring or cutting techniques; instead, the incision is dependent upon the length of cutting edge 48a. Thus, the length of the arteriotomy is controlled by the distance between tip 48d and neck 47, otherwise referred to as the horizontal length of the foot. In the preferred embodiment shown in FIG. 4, the length L is approximately 4 mm.

Referring to FIG. 1, handle 30 is designed such that, when held, the force F applied to button 61 actuator is out of plane with cutting plane C defined by the contact point of moving blade 50 and stationary blade 40. As such, when actuated, the force due to actuating is not in line with the cutting plane and does not produce a moment that causes cutting edge 48a or leading edge 53a to cut tissue inadvertently. Similarly, any steadying counterforce applied by the surgeon is also out of plane with the plane of the cutting edge and any moment produced by the steadying counterforce is also out of plane with the cutting edge. Further, because the actuation motion is perpendicular to the cutting motion, inadvertent cutting is prevented during piercing action.

This inventive design is in contrast to a more typical prior art arteriotomy device, where the actuation force is typically in line with the cutting action, and therefore the user may inadvertently cut tissue by the cutting blade. It is important to avoid inadvertent cutting as to do so might create an incision of the wrong length. As discussed above, precise incisions are necessary when employing automatic anastomotic devices to connect bypass grafts to blood vessels.

Further, when foot 48 of device 20 is disposed within the blood vessel, shoulder 46 butts up against the adventitia or outer surface of the blood vessel and prevents foot 48 from unnecessarily contacting the inner surface of the blood vessel. Shoulder 48 also serves to steady device 20 when actuation force F is applied to button 61.

In an alternate embodiment depicted as device 120 in FIG. 14, moving blade 150 can be formed to eliminate the need for spring 63. Moving blade 150 is preferably a resilient sheet of metal, fixed on a proximal end to handle 130. Button 161 includes a stop 162 which retains button 161 within handle 130. When button 161 is actuated, moving blade 150 straightens to extend distally relative to stationary blade 140. When the surgeon releases button 161, the spring force created by resilient moving blade 150 returns button 161 to its initial position and returns the distal end of moving blade 150 to a position proximal of stationary blade 140.

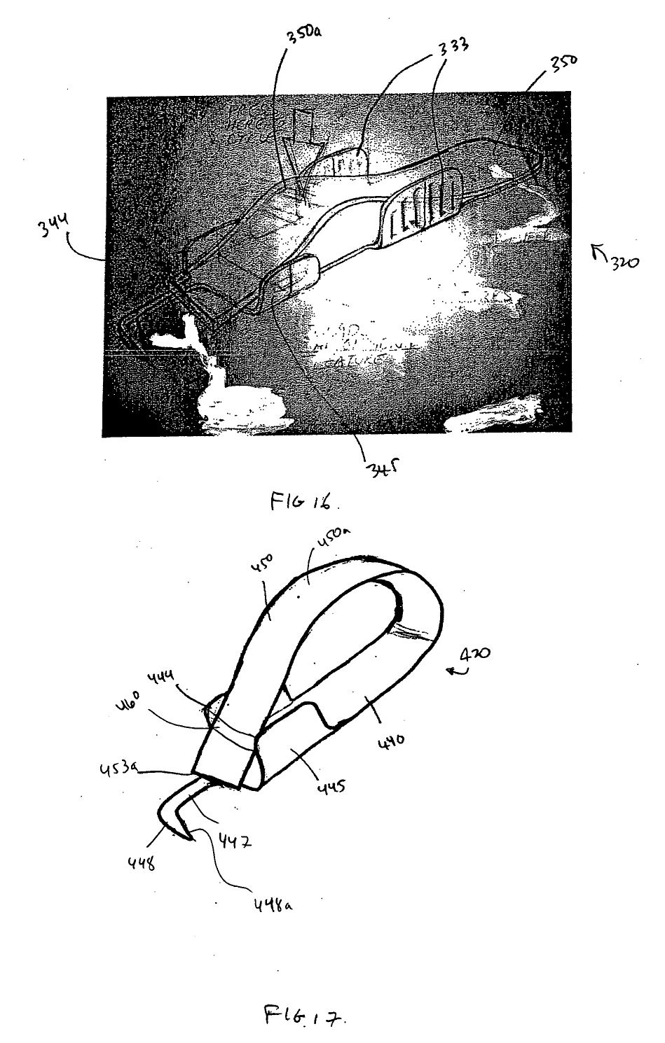

A second alternate embodiment is depicted as device 220 at FIGS. 15(a) and 15(b), and includes a moving blade 250 formed to eliminate the need for actuation button 61 and spring 63. In this embodiment, moving blade 250 is disposed within and constrained by handle 230. Moving blade has a proximal portion 250a which is retained within the proximal portion of handle 230, a middle portion 250b, connected to proximal portion 250a, bent to be disposed outside handle 230 in a slot formed in handle 230, and a distal portion 230c which acts in concert with stationary blade 240 to cut blood vessel tissue as described above. Moving blade 250 is actuated by pushing on middle portion 250b, which straightens moving blade 250 within handle 230 to extend distal portion 250c distally relative to stationary blade 140 and cut tissue disposed therebetween.

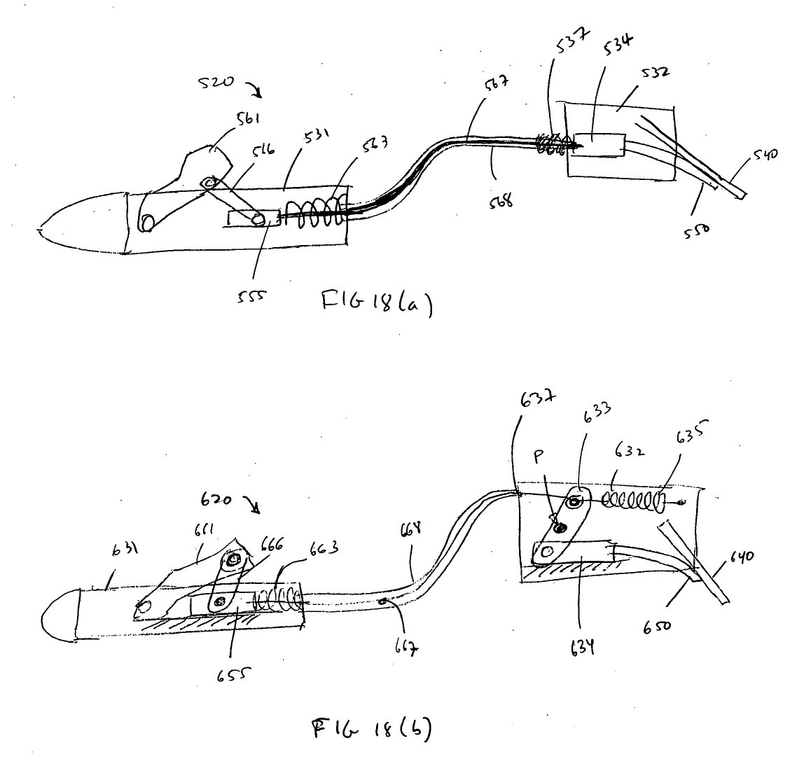

A third alternate embodiment, depicted as device 320 at FIG. 16, can be constructed from sheet metal, similar to traditional tweezers. As such, device 320 eliminates the need for the handle. Device 320 includes a stationary blade 340 that has blade guides 344 and 345 that extend orthogonally from either side of stationary blade 340 at a distal location, and gripping portions 333 that extend orthogonally from either side of stationary blade 340 at a proximal location. Moving blade 350 is spot welded to stationary blade 340 at a proximal location, and is formed such that moving blade 350 includes a hump 350a. When a force is applied to hump 350a, moving blade 350 moves distally relative to stationary blade 340 to cut tissue disposed therebetween, as described above in connection with the other embodiments. Once the tissue is cut, the force is removed from hump 350a, and moving blade 350 moves back to its original position due to the resilient nature of moving blade 350.

In a fourth embodiment, depicted as device 420 at FIG. 17, is even further simplified. In this embodiment, moving blade and stationary blade are formed from the same piece of sheet metal such that a moving blade 450 is connected to a stationary blade 440, having a neck 447 connected to a foot 448. Guides 444 and 445 extend orthogonally from the distal end of stationary blade 440. A band 460 may be fashioned to retain moving blade 450 within guides 444, 445. Device 420 is designed to be grasped within the palm of the surgeon and utilized in a similar manner as those embodiments described above. Moving blade 450 is actuated by applying a force to an intermediate portion 450a, thereby forcing leading edge 453a to move toward cutting edge 448a. As with device 320, once the force is removed, moving blade 450 moves back to its original position due to the resilient nature of moving blade 450.

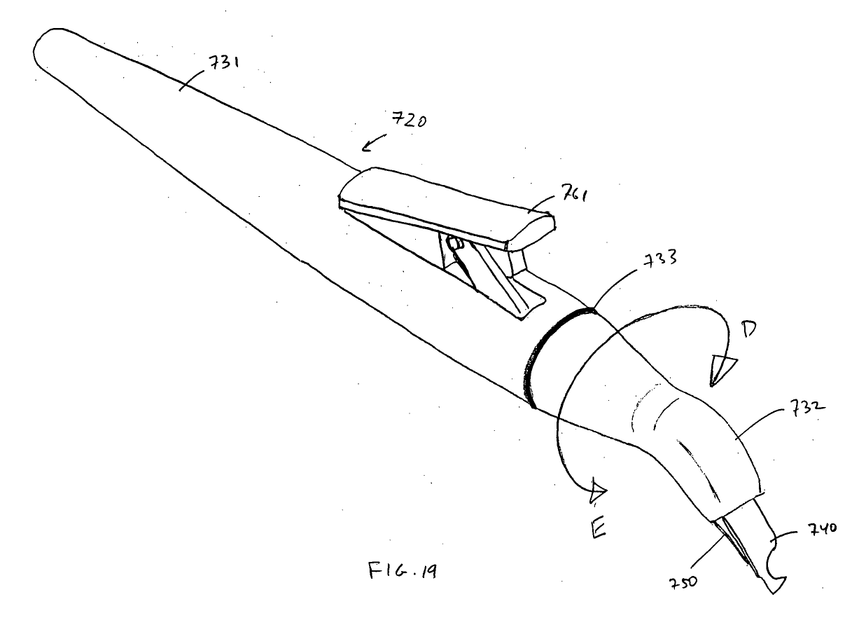

A fifth embodiment is depicted as devices 520, 620 in FIGS. 18(a) and 18(b), wherein proximal portion 531, 631 of handle 530, 630, can be connected to end effector 532, 632 by intermediate portion 533, 633. Intermediate portion 533, 633 may be malleable so as to permit the surgeon to actuate moving blade 550, 650 from a position remote of the incision site. For example, when anastomosing an artery on the posterior or inferior wall of the heart, it may be difficult for the surgeon to access the preferred incision site. Devices 520, 620 permit the surgeon to position proximal portion 531, 631 on one side of the heart, and then bend intermediate portion 533, 633 such that end effector 532, 632 is appropriately positioned to incise the vessel.

Alternatively, intermediate portion 533, 633 may be designed so as to permit device 520, 620 to be actuated from a position outside the chest cavity. In either case, intermediate portion 532, 632 may be designed similarly to the flexible connector described in U.S. patent application Ser. No. 60/551,609, filed on Mar. 9, 2004, a ball-and-socket type shaft as described in U.S. patent application Ser. No. 09/492,558, filed on Jan. 27, 2000, or of the type described in U.S. patent application Ser. No. 10/736,199, filed on Dec. 15, 2003 (Attorney Docket No. ETH 5099), the disclosures of which are hereby incorporated by reference.

The embodiments shown in FIGS. 18(a) and 18(b) are substantially similar. Each device 520, 620 includes a stationary blade 540, 640 and a moving blade 550, 650 that moves relative to stationary blade 540, 640 to cut tissue between the blades as described in connection with the above embodiments. Proximal sections 531, 631 of the embodiments each contain an actuator button 561, 661 pivotably connected to a link 566, 666, which in turn is pivotably connected to a slide 555, 655 that moves through a defined pathway within proximal sections 531, 631 and acts against a spring 563, 663.

The embodiments of FIGS. 18(a) and 18(b) differ primarily in the design of the end effector 532, 632 and how they are actuated. Slide 555 of device 520 is connected to a semi-rigid rod 567 at the proximal end of rod 567. Rod 567 is movable within a sheath 568, and may be a plastic rod or a wound coil spring or any other device having a stiffness that permits rod 567 to be pushed within sheath 568. Slide 655 of device 620 is connected to a cable 667 at the proximal end of cable 667. Cable 667, like rod 567, is movable within a sheath 568, and may be a plastic rod or a wound coil spring. Cable 667, however, does not need to have a stiffness that permits cable 667 to be pushed within sheath 668.

End effector 532 includes a slide 534, which is operatively connected on its proximal end to the distal end of cable 567, and on its distal end to movable blade 550. End effector 632 also includes a slide 634 that is operatively connected on its distal end to movable blade 550. End effector 632, however, also includes a link 633 which is operatively connected on one end to the distal end of cable 667, is pivotable about a pivot point P attached to the housing of end effector 632, and is pivotably connected on its other end to slide 634. Link 633 is also connected to a spring 635 which supplies a force that biases slide 634 and therefore moving blade 650 in their proximal, initial positions. An alternate means of connecting end effector 532, 632 is to connect the distal end of sheath 567, 667 to the housing of end effector 532, 632 via a connector 537, 637. Connector 537, 637 permits end effector 532, 632 to rotate or articulate or both relative to sheath 567, 667 so as to position end effector 532, 632 at different orientations.

Referring to FIG. 18(a), when device 520 is actuated by pressing on button 561, slide 555 moves distally to act against spring 563 and pushes cable 567, which in turn pushes slide 534 and moving blade 550 relative to stationary blade 540 to thereby cut tissue. While this design is simple, it is much more difficult to push cable 567 over any length than to pull cable 567. Referring to FIG. 18(b), when device 620 is actuated by pressing on button 661, slide 655 moves proximally through a defined pathway within proximal portion 631 and acts on spring 663. In turn, slide 655 pulls cable 667, which pulls link 633, to overcome spring force 632. When this occurs, link 633 pivots about pivot point P and pushes slide 634 and moving blade 650 relative to stationary blade 640 to thereby cut tissue. Those skilled in the art can devise other means of remotely actuating moving blade based on these principals.

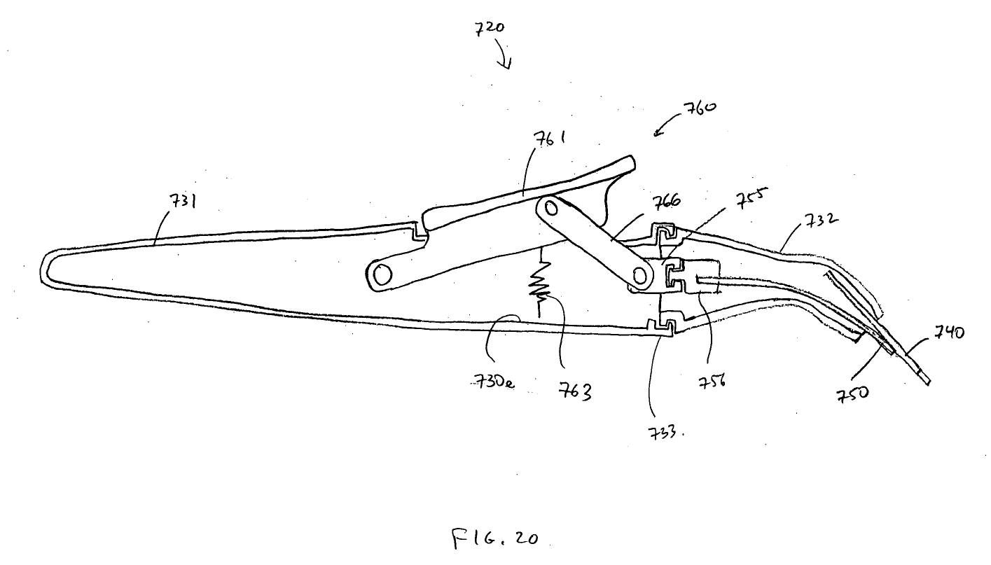

A sixth embodiment is depicted as device 720 in perspective in FIG. 19 and in cross section in FIG. 20. Device 720 is configured and operates similarly to device 20 except that distal portion 732 is rotatable with respect to proximal portion 731 in a clockwise or counterclockwise direction as indicated by arrows D and E. Proximal portion 731 is rotatably connected to distal portion 732 at coupling 733. In the embodiment shown in FIG. 20, distal portion 732 rotates within proximal portion 731. Alternatively, proximal portion 731 can be configured to rotate within distal portion 732. Those skilled in the art can configure coupling 733 in many known ways.

As with prior embodiments, device 720 includes an actuator 760 that has a button 761 pivotably mounted to proximal portion 731 of handle 730, a spring 763 (shown schematically) disposed between button 761 and an inner surface 730e of handle 730, a slide 755 for moving in a longitudinal direction, and a link 766 pivotably connected at a link proximal end to the distal end of button 761 and on a link distal end to slide 755. Slide 755, however, is formed as a proximal part of a cylindrical coupling that is rotatably coupled to a connector 756 which acts as the distal part of the cylindrical coupling. Connector 756, in turn, is connected to moving blade 750. Slide 755 and connector 756 are slidable as a unit within a path molded within handle 730. The path may extend between proximal portion 731 and distal portion 732.

Thus, as with earlier embodiments, moving blade 750 is moved relative to stationary blade 740 when button 761 is depressed as slide 755 and connector 756 act as one unit to slide within the path. The surgeon, however, has the option of rotating distal portion 732 relative to button 761 and proximal portion 731 to better access the planned incision site. Coupling 733 may include detents (not shown) so that distal portion 732 can be rotated in predetermined increments. Coupling 733 may also include a lock (not shown) to lock distal portion 732 with respect to proximal portion 731 prior to actuating device 720.

Specific construction details that are not shown are believed to be within the purview of those of ordinary skill in the art. The present invention has been described herein with reference to certain preferred embodiments. These embodiments are offered as illustrative, and not limiting, of the scope of the invention. Certain modifications or alterations may be apparent to those skilled in the art without departing from the scope of the invention, which is defined by the appended claims.

1. A device for incising a blood vessel, comprising:
a handle having a longitudinal axis;
a stationary blade having an upper surface and extending from the distal end of the handle at an angle out of line with respect to the longitudinal axis;
a moving blade having a distal portion that includes a leading edge, the moving blade being slidable with respect to the stationary blade, the distal portion of the moving blade being substantially aligned with the stationary blade, the moving blade having a first position and being movable to a second position to cut tissue disposed between the upper surface of the stationary blade and the leading edge of the moving blade.

2. The device of claim 1, wherein the handle has at least one gripping portion.

3. The device of claim 1, wherein the stationary blade includes a body, a foot, and a neck that connects the body to the foot.

4. The device of claim 3, wherein the body is at least partially disposed within the handle.

5. The device of claim 1, comprising an actuator attached to the handle and operatively connected to the moving blade.

6. The device of claim 5, comprising a link pivotably connected to the actuator.

7. The device of claim 5, comprising a slide operatively connected to the actuator, the moving blade being connected to the slide.

8. The device of claim 3, wherein the foot includes a heel having a rounded surface.

9. The device of claim 3, wherein the foot includes a heel having a secondary cutting edge.

10. The device of claim 9, wherein the foot comprises a bottom surface, and the secondary cutting edge is angled and extends from the neck to the bottom surface.

11. The device of claim 3, wherein the foot comprises a bottom surface, and the width of the neck is less than any height measured from the bottom surface to the cutting edge.

12. The device of claim 1, wherein the stationary blade comprises a first guide and a second guide that extend orthogonally from the stationary blade.

13. The device of claim 1, wherein the stationary blade comprises a first guide and a second guide, each having a height that is at least substantially equal to the thickness of the moving blade.

14. The device of claim 1, wherein one of the moving blade and the stationary blade is bent so as to be in slidable contact with the other of the moving blade and the stationary blade when the moving blade is moved relative to the stationary blade.

15. The device of claim 1, wherein the stationary blade includes a sharp tip that is bent so as to be in slidable contact with the moving blade.

16. The device of claim 1, wherein the stationary blade includes a shoulder extending from the neck.

17. The device of claim 1, wherein a cutting angle formed by the cutting surface and the leading edge of the moving blade remains constant when moving blade moves from the first position to the second position.

18. The device of claim 17, wherein the cutting angle ranges from 14 degrees to 24 degrees.

19. The device of claim 17, wherein the cutting angle is approximately 19 degrees.

20. The device of claim 2, wherein the moving blade is attached to the handle and is formed to resiliently return to the first position from the second position.

21. The device of claim 2, wherein the handle comprises a proximal portion, and a distal portion rotatably connected to the proximal portion.

22. The device of claim 1, comprising a handle, a distal housing configured to contain at least a portion of the stationary blade and the moving blade, and a malleable portion connecting the handle to the distal housing.

23. A device for incising a blood vessel, comprising:
a handle having a longitudinal axis;
a moving blade operatively connected to the handle and having a leading edge, the moving blade being slidable with respect to the handle between a first position and a second position, the moving blade leading edge defining a cutting plane; and
an actuator attached to the handle and operatively attached to the moving blade, the actuator movable between a first position and a second position, the actuator designed to be responsive to a force applied substantially orthogonally to the cutting plane to move the moving blade from the first position to the second position.

24. The device of claim 23, wherein the moving blade extends from the distal end of the handle at an angle out of line with respect to the longitudinal axis.

25. The device of claim 23, comprising a stationary blade that extends from the distal end of the handle at an angle out of line with respect to the longitudinal axis.

26. The device of claim 23, comprising a stationary blade attached to the handle, the stationary blade having a neck and a foot connected to the neck, the foot having a cutting edge along an upper surface.

27. The device of claim 26, wherein the moving blade is relatively blunt, and the moving blade and the stationary blade are designed to cut tissue disposed between the upper surface of the foot and the leading edge of the moving blade when the moving blade moves from the first position to the second position.

28. A device for incising a blood vessel, comprising:
a handle having a proximal portion defining a longitudinal axis, and a distal portion extending from the proximal portion at an acute angle as measure from the longitudinal axis;
a stationary blade disposed at least partially within the distal portion of the handle; and
a moving blade operatively connected to the handle and being disposed at least partially within the distal portion of the handle, the moving blade being slidable between a first position and a second position, the moving blade being substantially aligned with the distal portion of the handle.
